(19) United States
(12) Patent Application Publication
(10) Pub. No.: US 2019/0054234 A1
(43) Pub. Date: Feb. 21, 2019

(54) ELECTRONIC CONTROL OF DRUG ADMINISTRATION VIA HYPODERMIC NEEDLE DEVICES

(71) Applicant: QUALCOMM Incorporated, San Diego, CA (US)

(72) Inventors: Robert GANTON, San Diego, CA (US); Robert BALLAM, Eatons Hill (AU); James PIERONEK, San Diego, CA (US); Paul Robert HOFFMAN, San Diego, CA (US)

(21) Appl. No.: 16/104,038

(22) Filed: Aug. 16, 2018

Related U.S. Application Data
(60) Provisional application No. 62/547,075, filed on Aug. 17, 2017.

(51) Int. Cl. A61M 5/172; A61M 5/158

(52) U.S. Cl. CPC A61M 5/172; A61M 5/158; A61M 2205/3368; A61M 2205/3379; A61M 2005/1588; A61M 2205/50; A61M 2205/609; A61M 2205/3592; A61M 2205/3331

(57) ABSTRACT

Disclosed are hypodermic needle devices with a dose dispensing mechanism and methods of using those hypodermic needle devices. The dose dispensing mechanism, which may be an electronically-controlled pump, may be used to measure out a specific dosage amount of a drug while compensating for environmental factors, such as temperature and pressure. This allows the device to be used by patients for self-administration since a specific dosage amount of the drug may be precisely delivered regardless of the environmental conditions at the time of delivery. The specific dosage amount may be set using the device or remotely, and a fingerprint sensor may be used to prevent injecting the drug into the wrong patient.

### CROSS-REFERENCE TO RELATED APPLICATIONS

This application claims the benefit of U.S. Provisional Application No. 62/547,075, filed Aug. 17, 2017, entitled “Electronic Control of Drug Administration via Hypodermic Needle Devices” which is incorporated herein by reference in its entirety.

### BACKGROUND

### Background Field

The subject matter disclosed herein relates to hypodermic needle devices (e.g., auto injectors, syringes, etc.), and more particularly to methods and apparatuses for electronically controlling the administration of drugs via hypodermic needle devices, in order to precisely control the amount of drug dispensed and restrict the delivery of that drug to the correct patient.

### Relevant Background

In the field of drug administration, it is often desirable to control drug administration to a patient. In particular, it may be desirable to ensure that the correct dosage of the drug is being dispensed to the patient. Complications may occur if the patient is receiving an incorrect or inaccurate dosage of a drug. Furthermore, it may be desirable to ensure that the drug is being dispensed to the correct patient.

However, this level of control may be difficult to attain in situations where the drug is dispensed by the patient (e.g., self-administered) rather than by a healthcare provider. For instance, patients may sometimes self-administer drugs by injecting themselves using simple devices (e.g., hypodermic needles) which have no capabilities of ensuring that the drug has been delivered properly (e.g., in the correct amount, and the correct drug to the correct patient). Thus, there exists a need for ways to precisely control the amount of a drug dispensed to a patient while restricting the delivery of that drug to the correct patient.

### SUMMARY

Embodiments disclosed herein are directed to hypodermic needle devices that have a dose dispensing mechanism and methods of using those hypodermic needle devices. In order to deliver to the user a specific dosage amount of a drug, the dose dispensing mechanism may be used to measure out an adjusted dosage volume of the drug in order to compensate for environmental factors such as temperature and pressure. There may be multiple methods of delivery of the specific dosage amount of the drug into the user. In some embodiments, the dose dispensing mechanism may be an electronically-controllable pump. The pump may be configured to displace a small, known volume of drug with each stroke of the pump. The hypodermic needle device may have a processor that may operate and control the pump for a certain number of strokes in order to measure out the desired adjusted dosage volume of drug to deliver to the user. This ensures that the specific dosage amount of the drug is precisely delivered to the user regardless of the environmental conditions at the time of delivery.

In some embodiments, the hypodermic needle device may have a dose dispensing mechanism that includes an electronically-controllable pump, and the electronically-controllable pump may include a piston actuated by a shaft. The pump is used to measure out the specific dosage amount of the drug into a reservoir chamber. Once the user has inserts the hypodermic needle device in the appropriate location, the user may then manually or electronically actuate the shaft of the dose dispensing mechanism (e.g., by depressing a button) in order to drive the piston and expel the drug from the reservoir chamber and into themselves. In some embodiments, the pump itself may be the dose dispensing mechanism. Once the user injects themselves with the hypodermic needle device, the pump may be used to directly pump the specific dosage amount of the drug into the user.

In some embodiments, the hypodermic needle devices may have fingerprint sensors that may be used for fingerprint validation in order to drive the dose dispensing mechanism. Any user that attempts to dispense the drug but is not authorized to receive the drug (e.g., the user is not the correct patient) will be locked out to ensure that the correct patient is using the hypodermic needle device and receiving the drug. For instance, if the dose dispensing mechanism includes a shaft that is electronically actuated by an actuator and the user supplies the wrong fingerprint to the fingerprint sensor, the actuator may be prevented from driving the shaft to expel the drug from the reservoir chamber. As another example, in embodiments where the pump is the dose dispensing mechanism, the pump may be prevented from operating if the user supplies the wrong fingerprint to the fingerprint sensor.

Embodiments disclosed herein are also directed to a method of dispensing a drug with a hypodermic needle device. In some embodiments, the method may include obtaining, by the hypodermic needle device, a specific dosage amount of the drug to dispense. In some embodiments, the method may further include determining, by the hypodermic needle device, one or more environmental factors. One of the one or more environmental factors may include an ambient temperature. In some embodiments, the method may include determining, by the hypodermic needle device, one or more environmental factors. One of the one or more environmental factors may include an ambient temperature. In some embodiments, the method may include determining, by the hypodermic needle device, an adjusted dosage volume from the specific dosage amount based on the one or more environmental factors. In some embodiments, the method may include determining, by the hypodermic needle device, a number of strokes to operate a pump of the hypodermic needle device based on the adjusted dosage volume and a stroke volume of the pump. In some embodiments, the method may include operating, by the hypodermic needle device, the pump for the determined number of strokes to transfer the adjusted dosage volume of the drug.

In various embodiments, operating the pump for the determined number of strokes serves to transfer the adjusted dosage volume of the drug into a reservoir chamber of the hypodermic needle device. In various embodiments, the method may further include dispensing, by the hypodermic needle device, the adjusted dosage volume of the drug in the reservoir chamber. In various embodiments, operating the pump for the determined number of strokes serves to dispense the adjusted dosage volume of the drug from the hypodermic needle device. In various embodiments, the pump is a multi-chambered pump having a plurality of chambers. In various embodiments, the method may further include selecting, by the hypodermic needle device, a chamber of the plurality of chambers of the multi-chambered pump based on the adjusted dosage volume and a stroke volume associated with the chamber. In various embodiments, the chamber of the plurality of chambers of the multi-chambered pump is selected to minimize the number of strokes. In various embodiments, the one or more environmental factors includes a density of the drug at a current temperature. In various embodiments, the one or more environmental factors includes a density of the drug at a current pressure. In various embodiments, the specific dosage amount of the drug is obtained from a third-party device.

Embodiments disclosed herein are also directed to a hypodermic needle device. In some embodiments, the hypodermic needle device may include a drug cartridge containing a drug. In some embodiments, the hypodermic needle device may include a reservoir configured to hold the drug prior to injection. In some embodiments, the hypodermic needle device may include a dose dispensing mechanism configured to transfer an amount of the drug contained in the drug cartridge to the reservoir. In some embodiments, the hypodermic needle device may include a sensor for measuring one or more environmental factors. One the one or more environmental factors may include an ambient temperature. In some embodiments, the hypodermic needle device may include a processor.

In some embodiments, the hypodermic needle device may include a non-transitory computer readable memory storing instructions that, when executed by the processor, cause the processor to determine a specific dosage amount of the drug to dispense. The instructions may also cause the processor to determine the one or more environmental factors from the sensor. The instructions may also cause the processor to determine an adjusted dosage volume from the specific dosage amount based on the one or more environmental factors. The instructions may also cause the processor to operate the dose dispensing mechanism to transfer the adjusted dosage volume of the drug.

In various embodiments, operating the dose dispensing mechanism serves to transfer the adjusted dosage volume of the drug from the drug cartridge into the reservoir. In various embodiments, the instructions, when executed by the processor, further cause the processor to dispense the adjusted dosage volume of the drug in the reservoir. In various embodiments, operating the dose dispensing mechanism serves to dispense the adjusted dosage volume of the drug from the hypodermic needle device. In various embodiments, the dose dispensing mechanism is an electronically-controlled pump. In various embodiments, the instructions, when executed by the processor, further cause the processor to determine a number of strokes to operate the pump based on the adjusted dosage volume and a stroke volume of the pump, and to operate the pump for the determined number of strokes to transfer the adjusted dosage volume of the drug. In various embodiments, the hypodermic needle device further includes a needle configured for dispensing the drug contained in the reservoir. In various embodiments, the one or more environmental factors includes a density of the drug at a current temperature. In various embodiments, the one or more environmental factors includes a density of the drug at a current pressure. In various embodiments, determining the specific dosage amount of the drug includes obtaining the specific dosage amount from a third-party device.

Embodiments disclosed herein are also directed to a hypodermic needle device. In some embodiments, the hypodermic needle device may include means for obtaining a specific dosage amount of the drug to dispense. In some embodiments, the hypodermic needle device may include means for determining one or more environmental factors. One of the one or more environmental factors may include an ambient temperature. In some embodiments, the hypodermic needle device may include means for determining an adjusted dosage volume from the specific dosage amount based on the one or more environmental factors. In some embodiments, the hypodermic needle device may include means for transferring the adjusted dosage volume of the drug.

In various embodiments, the hypodermic needle device may further include means for determining a number of strokes to operate a pump of the hypodermic needle device based on the adjusted dosage volume and a stroke volume of the pump, and means for operating the pump for the determined number of strokes to transfer the adjusted dosage volume of the drug. In various embodiments, the pump is a multi-chambered pump having a plurality of chambers. In various embodiments, the hypodermic needle device may further include means for selecting a chamber of the plurality of chambers of the multi-chambered pump based on the adjusted dosage volume and a stroke volume associated with the chamber. In various embodiments, the one or more environmental factors includes a density of the drug at a current temperature. In various embodiments, the one or more environmental factors includes a density of the drug at a current pressure. In various embodiments, the specific dosage amount of the drug is obtained from a third-party device.

Embodiments disclosed herein are also directed to a non-transitory computer readable medium. In some embodiments, the non-transitory computer readable medium contains instructions that, when executed by a processor, cause the processor to obtain a specific dosage amount of the drug to dispense. In some embodiments, the instructions, when executed by a processor, further cause the processor to determine one or more environmental factors, wherein one of the one or more environmental factors includes an ambient temperature. In some embodiments, the instructions, when executed by a processor, further cause the processor to determine an adjusted dosage volume from the specific dosage amount based on the one or more environmental factors. In some embodiments, the instructions, when executed by a processor, further cause the processor to determine a number of strokes to operate a pump of the hypodermic needle device based on the adjusted dosage volume and a stroke volume of the pump. In some embodiments, the instructions, when executed by a processor, further cause the processor to operate the pump for the determined number of strokes to transfer the adjusted dosage volume of the drug.

In various embodiments, the one or more environmental factors includes a density of the drug at a current temperature. In various embodiments, the one or more environmental factors includes a density of the drug at a current pressure.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is an example system for providing information about the administration of medicine by a hypodermic needle device to one or more stakeholders.

FIG. 2 is an illustration of an example hypodermic needle device, according to an embodiment.

FIG. 3 is a block diagram illustrating the of components of a hypodermic needle device, according to an embodiment.

FIG. 4 is a simplified cross-sectional diagram of an embodiment of a hypodermic needle device.

FIG. 5 is a flow diagram illustrating a method of dispensing a drug with a hypodermic needle device, according to an embodiment.

FIG. 6 is a simplified cross-sectional diagram of an embodiment of a hypodermic needle device.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

Hypodermic needle devices (e.g., injector pens, auto injectors, syringe needles, etc.) dispense liquid drugs into the body of a patient (e.g., directly into a muscle, vein, or other location under a patient's skin) by pushing a volume of the drug from the chamber or cylinder within the device through a hypodermic needle that has been injected into the skin of patient. Often, these drugs may be self-administered by the patient, such as when the drugs are administered in an emergency (as may be the case with epinephrine, for example) or frequently administered (as may be the case with insulin, for example).

Establishing that the right dose of the right drug is dispensed to the right patient at the right time via the right route may be important not only to the person taking the drug, but to many other entities as well. Other stakeholders that have an interest in this information include, for example, the doctor that prescribed and/or is overseeing the administration of the drug, a manufacturer of the drug, an insurance provider (and/or other payer), a government health agency and/or other health organization, and/or the like. For each of the stakeholders, the use and/or misuse of a drug may impact the decisions of a stakeholder with regard to the drug. For example, a drug that is consistently misused may impact whether or how an insurance provider is willing to pay for the use of the drug, and/or how to adjust premiums for patients that may consistently misuse drugs. It may also prompt a manufacturer to modify the means of administration of the drug to help reduce the misuse of its administration. All stakeholders may be impacted by use/misuse information in determining how effective a drug for a population of patients.

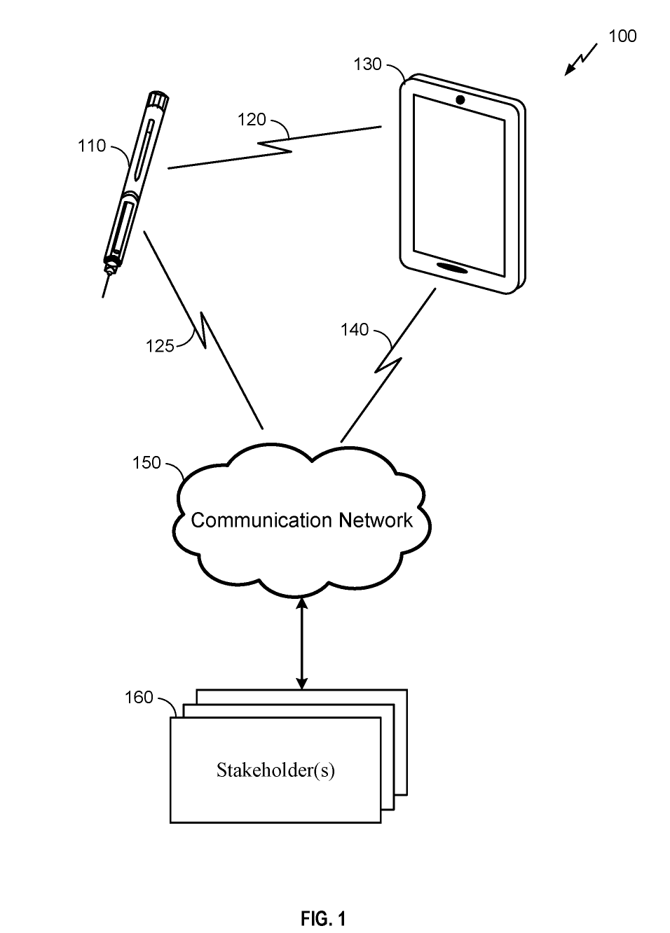

FIG. 1 is an example system 100 for providing information about the administration of medicine by a hypodermic needle device 110 to one or more stakeholders 160. Here, the system 100 may comprise the hypodermic needle device 110 as described herein, along with a connecting device 130, communication network 150, and the stakeholder(s) 160. It will be understood, however, that embodiments of a system 100 may include a different configuration of components, the addition and/or omission of various components, and/or the like, depending on desired functionality. Moreover, it will be understood that techniques described herein may be utilized in a hypodermic needle device 110 that may not necessarily be part of a larger system, such as the system 100 illustrated in FIG. 1.

The hypodermic needle device 110, which is described in more detail herein below, is used to dispense a drug to a patient. Here, a person (e.g., a doctor, nurse, or patient him/herself) may dispense the drug by engaging a physical mechanism (e.g., pressing down on a plunger, actuating automatic injection, etc.) while a needle of the hypodermic needle device 110 is inserted into the patient's skin. In some embodiments, once the drug is dispensed, the hypodermic needle device 110 may register, store and transmit data associated with the administration of the drug to the connecting device 130. This data may be transmitted wirelessly via a wireless communication link 120, using any of a variety of wireless technologies as described in further detail below. That said, some embodiments may additionally or alternatively utilize wired communication.

The connecting device 130 may comprise any of a variety of electronic devices capable of receiving information from the hypodermic needle device 110 and communicating information to the stakeholder(s) 160 via the communication network 150. This may include, for example, a mobile phone, tablet, laptop, portable media player, personal computer, or similar device. In some embodiments, the connecting device 130 may comprise a specialized device utilized for purposes of conveying information from the hypodermic needle device 110 (and possibly other medical devices) to the stakeholder(s) 160.

The connecting device 130 may execute an application to provide the data processing and/or relaying functionality illustrated in FIG. 1. In some embodiments, the application may be configurable by a user, or may simply be downloaded to the connecting device 130 and executed automatically. The application may help establish the communication link 120 between the hypodermic needle device 110 and the connecting device 130, which may or may not require input from the user, depending on desired functionality. In some embodiments, the application may provide instructions to a user on proper use of the hypodermic needle device 110 and/or feedback to a user when improper use of the hypodermic needle device 110 is detected. Additional and/or alternative functionality of an application executed by the connecting device 130 may be utilized as desired. (Such functionality may include simple relaying of the data to a remote destination or interacting with the patient about the drug administration such as confirmation and user feedback.)

The communication network 150 may comprise any of a variety of data communication networks, depending on desired functionality. The communication network 150 may include any combination of radio frequency (RF), optical fiber, satellite, and/or other wireless and/or wired communication technologies. In some embodiments, the communication network 150 may comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (Cat-M) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

The communication link 140 between the connecting device 130 and the communication network 150 may vary, depending on the technologies utilized by these components of the system 100. For embodiments where the connecting device 130 is a mobile phone, for example, the communication link 140 may comprise a wireless communication link utilizing the mobile phone's cellular or Wi-Fi® functionality. In embodiments where the connecting device 130 is a personal computer, the communication link 140 may comprise a wired communication link that accesses the medication network 150 via a cable or digital subscriber line (DSL) modem.

It may be noted that some embodiments may not utilize a connecting device 130 to relay data to the communication network 150. In such embodiments, the hypodermic needle device 110 may connect directly to the communication network 150 (as shown in FIG. 1 by communication link 125, which may be used in addition to or as an alternative to communication link 120). For example, the hypodermic needle device 110 may comprise a Long Term Evolution (LTE) category M (Cat-M) device, NarrowBand IoT (NB-IoT), or other Low Power Wide Area Network (LPWAN). Additionally or alternatively, the hypodermic needle device 110 may comprise wireless technology similar to the corresponding functionality of the connecting device 130 described above. In such embodiments, the communication network may additionally or alternatively comprise a Bluetooth Mesh network (such as CSRMesh), a Wi-Fi network, Zigbee, or WWAN (such as LTE, including Cat-M, or 5G). In some embodiments, the hypodermic needle device 110 may connect both with the communication network 150 via medication link 125 and with the connecting device 130 the communication link 120. In such embodiments, the connecting device 130 may not need to separately communicate information regarding the hypodermic needle device 110 to stakeholders 160, but instead the hypodermic needle device 110 may communicate this information directly to the stakeholders 160 via the communication network 150.)

As noted above, the stakeholder(s) 160 may include any of a variety of entities with an interest in the proper administration of medicine by the hypodermic needle device 110. This may include an individual practitioner (e.g., a doctor or nurse), a hospital, a drug manufacturer, an insurance provider (or other payer), a government agency or other health organization, and/or the like. In some embodiments, the user of the hypodermic needle device 110 (e.g., the patient) may also be a stakeholder 160 to which information regarding the use of the hypodermic needle device 110 is provided. Governmental health regulations and/or legal agreements between the patient and/or the stakeholder(s) 160 may apply to the dissemination of information regarding the administration of a drug by the hypodermic needle device 110 to the stakeholder(s) 160.

In some embodiments, the hypodermic needle device 110 may be provided with a value for a specific dosage amount of drug to dispense and/or the fingerprint profile of a patient prior to the hypodermic needle device 110 being used to dispense a drug. For example, the connecting device 130 may be used to supply the value of the specific dosage amount and/or the patient's fingerprint profile to the hypodermic needle device 110 via the communication link 120, or that information may be supplied to the hypodermic needle device 110 via the communication network 150 and/or the communication link 125. Digitally supplying the hypodermic needle device 110 with the specific dosage amount beforehand may obviate the need for a user to manually configure the specific dosage amount (e.g., by adjusting a dosage dial) prior to injection.

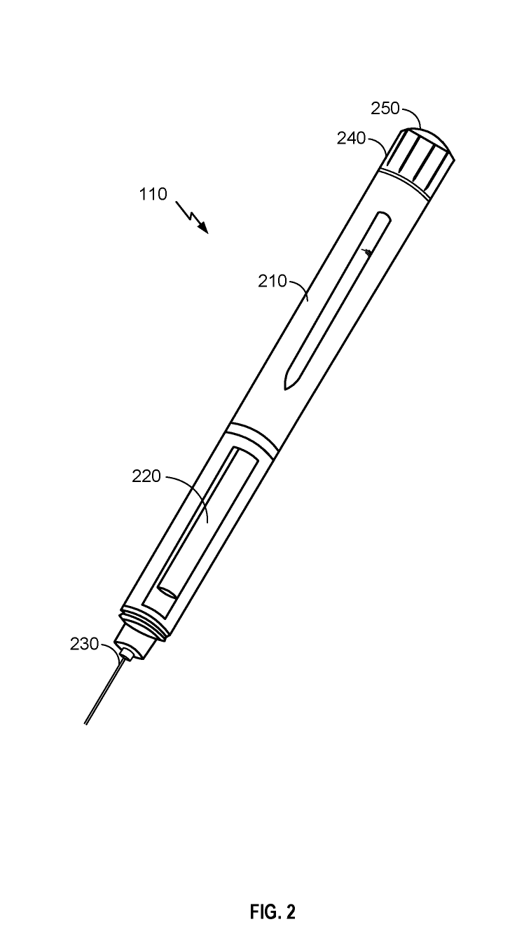

FIG. 2 is an illustration of an example hypodermic needle device 110, according to an embodiment. Here, a body 210 of the hypodermic needle device 110 may house dose dispensing and dose control mechanisms, including electrical and mechanical components, to ensure that a proper dosage of the drug is dispensed. In some embodiments, the components of a dose dispensing mechanism may include a plunger configured to displace a volume of the drug through the reservoir chamber 220 and out the needle assembly 230. Embodiments of a hypodermic needle device 110 include a dosage dial 240 that may be adjusted (e.g., by turning the dial clockwise or counterclockwise) to alter the dosage amount dispensed by the hypodermic needle device 110. The dosage may be dispensed by pressing the button 260, which may be coupled to a dose dispensing mechanism to control the dispensing of the drug.

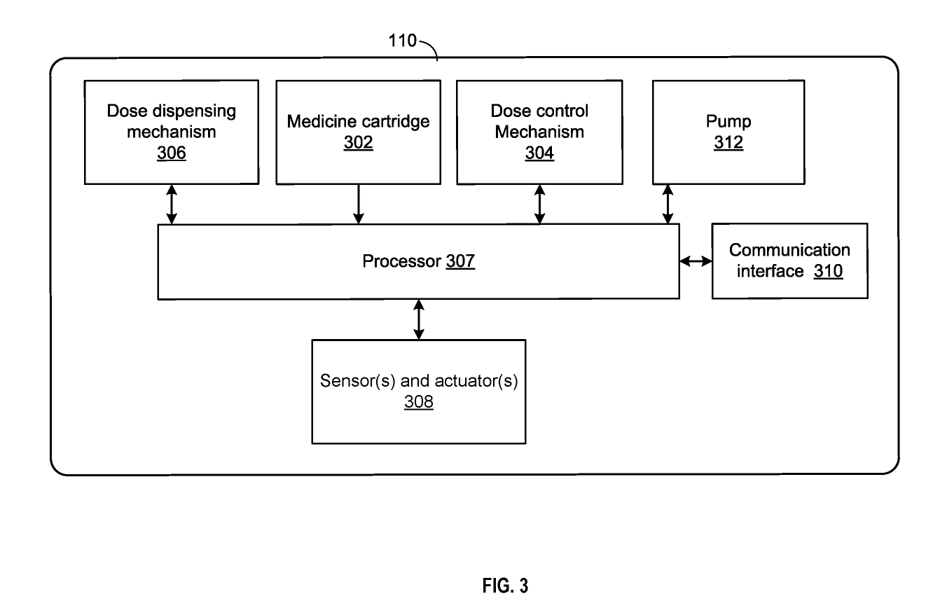

It will be understood however that the hypodermic needle device 110 illustrated in FIG. 2 is provided as a non-limiting example, according to an embodiment. Alternative embodiments may vary in size, shape, and/or other ways. A hypodermic needle device 110 may be described more generally as having various components is illustrated in FIG. 3.

FIG. 3 is a block diagram illustrating the of components of a hypodermic needle device 110, according to an embodiment. The hypodermic needle device 110 may include a housing (not shown) structured to hold a medicine cartridge 302, which may store medicine to be dispensed by the hypodermic needle device 110. In some embodiments, the medicine cartridge 302 may be referred to as a drug container and/or drug chamber.

The hypodermic needle device 110 may also include a dose control mechanism 304 to select or set a dose of the drug to be dispensed. For instance, the dose control mechanism 304 may be include a dosage dial (e.g., the dosage dial 240) that may be adjusted to alter the dosage amount dispensed by the hypodermic needle device 110. The user may adjust the dosage dial to a specific dosage amount, which will be considered by processor(s) of the hypodermic needle device 110 (e.g., when operating the pump 312 to measure out the specific dosage amount of the drug for dispensing. Thus, the volume of the drug dispensed may correspond to the selected dosage amount indicated by the dosage dial.

The hypodermic needle device 110 further includes a pump 312 for measuring out the specific dosage amount of the drug for dispensing. In some embodiments, the pump 312 may be any suitable, electronically-controllable micropump that is small but accurate. In some embodiments, the pump 312 may be a piezoelectric, micromachined silicon, or magnetic-based pump. In some embodiments, the pump 312 may be a small-volume doser that is actuated many times to deliver a full dose, with each actuation delivering a small, but very accurate, amount of the drug. In such embodiments, the pump 312 may be actuated thousands of times for the minimum dosing requirement to insure that accuracy of better than 0.1% is attained in dosing volume. The small actuation size of the pump 312 allows the power source that drives the pump 312 to be small and efficient and also allows for a safe drug delivery process by minimizing the peak pressures that may be applied to the patient. In some embodiments, the pump 312 may be a small mechanical pump which has very high accuracy and nullifies the error introduced by many of the error sources associated with traditional injector pens. In some embodiments, the pump 312 may be a low-power pump that is only capable of pumping very small amounts.

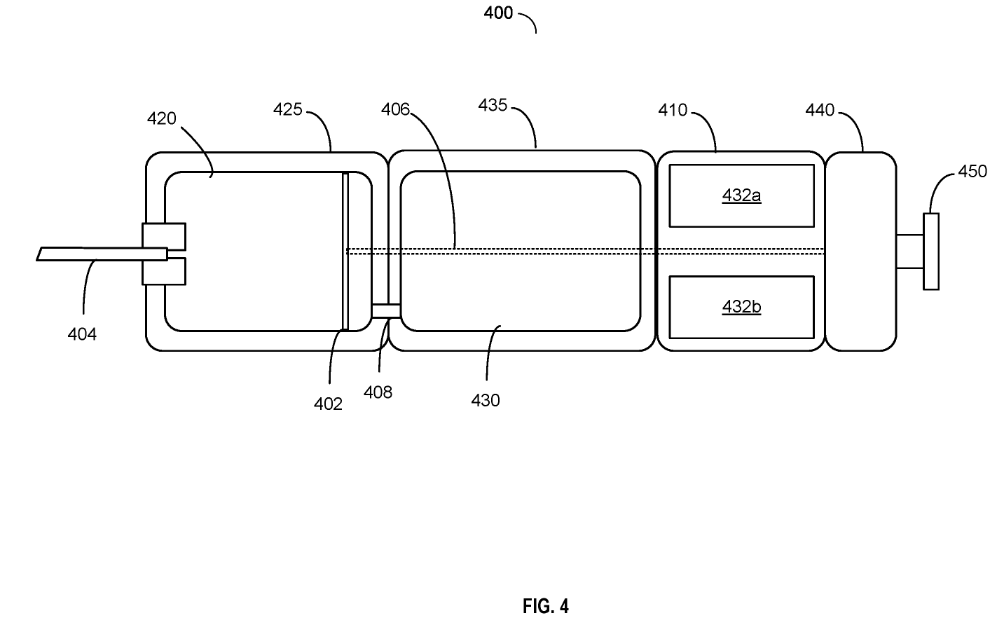

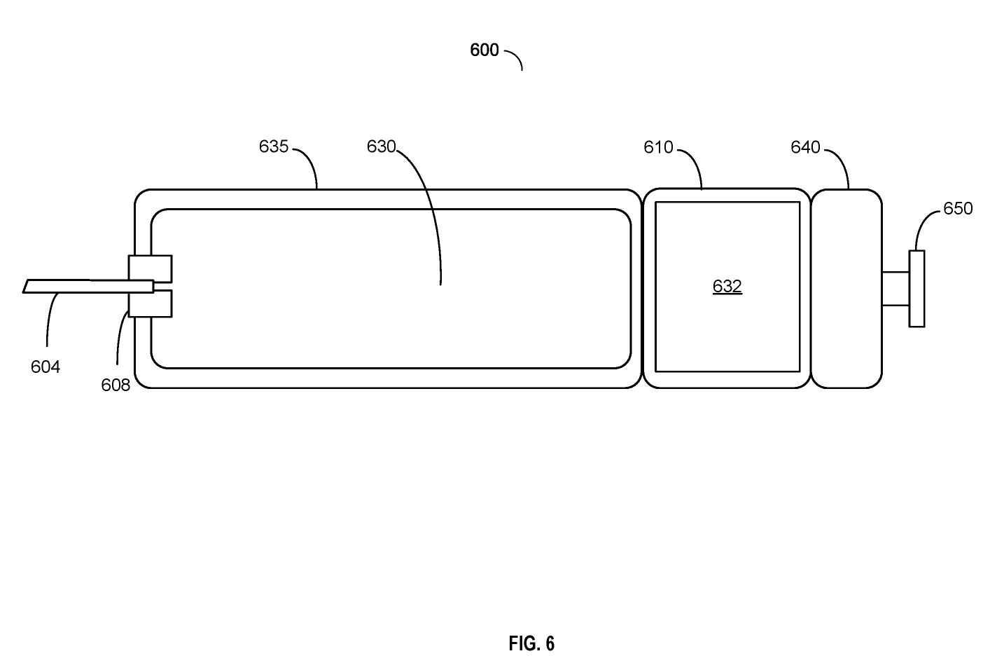

The hypodermic needle device 110 further includes a dose dispensing mechanism 306 to dispense a dose of the drug (e.g., from the medicine cartridge 302 or a reservoir), based on the dose selected or set by dose control mechanism 304. In some embodiments (e.g., such as the one shown in FIG. 4), the dose dispensing mechanism 306 may be a plunger (e.g., a piston and shaft) that is configured to dispense all of the drug contained in a reservoir, which may have been previously measured out into the reservoir using the pump 312. In some embodiments (e.g., such as the one shown in FIG. 6), the dose dispensing mechanism 306 may be the pump 312, which may be used to directly dispense a specific dosage amount of the drug into the user.

The hypodermic needle device 110 may include other devices to facilitate dispensing of medicine. In the example of FIG. 3, the hypodermic needle device 110 includes sensor(s) and actuator(s) 308. Additionally, the hypodermic needle device 110 can include a processor 307, which is communicatively coupled with the sensor(s) and actuator(s) 308 and configured to, among other things, control the operations of the actuator(s) based on the information collected by the sensor(s). For example, the sensors of sensor(s) and actuator(s) 308 may collect information of certain physical conditions at, for example, medicine cartridge 302, pump 312, dose control mechanism 304, and dose dispensing mechanism 306. Based on the collected information, the processor 307 may control the actuators of sensor(s) and actuator(s) 308 to change the operations of the pump 312, dose control mechanism 304, and/or dose dispensing mechanism 306. For example, based on fingerprint information received at a finger print sensor, the processor(s) 307 may control the operations of the pump 312 or the actuators associated with the dose dispensing mechanism 306 to prohibit administration of the drug.

In some embodiments, the dose control mechanism 304, the dose dispensing mechanism 306, and/or pump 312 may be in electronic communication with the sensor(s) and actuator(s) 308 via the processor 307. In particular, the pump 312 (which may be part of the dose dispensing mechanism 306) may be configured to displace a specific volume of the drug based on electronic inputs supplied from the processor 307. For example, the pump 312 may be is configured to displace 10 Pico liters (pL) of the drug per stroke of the pump 312. The processor 307 may be configured to execute a set of instructions for determining a specific dosage amount for the drug to dispense to the patient. Based on that specific dosage amount, processor 307 may control the pump 312 in order to measure out and displace the volume of the drug that corresponds to the specific dosage amount. For example, if each stroke of the pump 312 displaces 10 pL of the drug, the processor 307 may determine how many strokes of the pump are needed to displace the volume of the drug corresponding to the specific dosage amount. The processor 307 may then instruct the pump 312 to perform that many strokes in order to measure out the specific dosage amount of the drug. As an example, the processor(s) could operate the pump 312 for 100,000 strokes, with each stroke dispensing exactly 10 pL, to get an exact volume of drug dispensed. Thus, the number of strokes the pump 312 is actuated may be changed in order to arrive at a specific total amount of drug dispensed. In some embodiments, the processor 307 may be very good at counting the number of strokes of the pump 312 and actuating the low-powered pump 312 many times.

Furthermore, the processor 307 may be able to compensate for environmental factors and conditions, such as the temperature/pressure of the surrounding environment or the temperature/pressure of the fluid containing the drug. Since all of those factors may affect the density of the drug, a specific dosage amount of the drug may correspond to different volumes depending on environmental conditions. For instance, in the previous example, assume the processor 307 instructs the pump 312 to perform 100,000 strokes when the temperature is 22 degrees Celsius. At a higher temperature, such as 30 degrees Celsius, the density of the drug may be higher. Thus, the same equivalent mass of the drug may occupy more volume at higher temperatures. Alternatively, at a higher temperature, the density of the drug may be lower, and thus the same equivalent mass of the drug may occupy less volume at higher temperatures. The processor 307 may be able to obtain measurements for all these environmental factors (e.g., the sensor(s) and actuator(s) 308 may include temperature and pressure sensors that are in communication with the processor 307) and determine the number of strokes needed to supply the specific dosage amount of the drug at current conditions. For instance, the processor 307 may determine that 110,000 strokes are needed when the temperature is at 30 degrees Celsius. Thus, if environmental factors cause a volume variation in the drug, the processor(s) may compensate for environmental factors and conditions in order to dispense precisely the right amount of drug to the user regardless of temperature, pressure, and so forth.

In some embodiments, the pump 312 may be a pump with zero temperature coefficient, such that the volume pumped in each stroke does not change with temperature.

For instance, the pump 312 may be capable of pumping 10 pL each stroke regardless of temperature. However, in some embodiments, the pump 312 may not have a zero temperature coefficient. Instead, the processor(s) may compensate for any changes in the volume pumped in each stroke due to temperature. For instance, if the pump 312 pumped 10 pL per stroke at 20 degrees Celsius and 10.125 pL per stroke at 22 degrees Celsius, the processor(s) may be able to factor that into the calculation of the number of strokes to perform to obtain the correct volume of the drug.

Moreover, the hypodermic needle device 110 may include a communication interface 310 which may communicate using wireless and/or wired means (e.g., via wireless link 120 and/or 125 of FIG. 1). Communication interface 310 may enable transmission of information related to dispensing the drug, including an indication of whether the drug was dispensed properly or a report that the specific dosage amount of the drug was accurately dispensed. Additionally or alternatively, the hypodermic needle device 110 may communicate information related to a quantity of medicine to be dispensed, a quantity of medicine that has been dispensed, a quantity of medicine remaining in medicine cartridge 302, etc. The information may then be displayed to the user via an user interface, to assist the user in dispensing of the medicine.

FIG. 4 is a simplified cross-sectional diagram of an embodiment of a hypodermic needle device 400. The hypodermic needle device 400 may have a body 410 for housing dose dispensing and dose control mechanisms, including electrical and mechanical components, to ensure that a proper dosage of the drug is dispensed. In the figure, these components are shown as electronic units 432a and 432b, which may include sensors, actuators, processors, as well as communication interface circuitries. The sensors may collect operation information including, for example, the temperature and pressure surrounding the hypodermic needle device 110, the temperature and pressure of the drug (e.g., in a drug container 435), and so forth. The processors may be able to collect operation information from the sensors and consider that information in order to execute certain decisions (e.g., to send instructions for controlling the actuators).

The hypodermic needle device 400 may have a drug container 435 containing a drug chamber 430. The drug chamber 430 may contain a drug to be delivered to a user of the hypodermic needle device 400. The hypodermic needle device 400 may also have a reservoir container 425 containing a reservoir chamber 420. The reservoir chamber 420 may be fluidly coupled to the drug chamber 430 by a pump 408. The dose dispensing mechanism of the hypodermic needle device 400 may include a dispensing piston 402 within the reservoir chamber 420. The dispensing piston 402 may be mechanically coupled with a shaft 406. The shaft 406 may be coupled to a dose dispensing button 450. In some embodiments, the dispensing piston 402 may be manually depressed by the user or a caregiver. Pressing down on the dose dispensing button 450 may actuate the shaft 406 and cause the dispensing piston 302 to extend further into the reservoir chamber 420, displacing a volume of the drug contained in the reservoir chamber 420 out of the needle assembly 404. In some embodiments, the dispensing piston 402 may have a spring-assist mechanism to make it easier to fully extend the dispensing piston 302 into the reservoir chamber 420 and ensure that all of the drug in the reservoir chamber 420 is expelled. The spring may provide constant pressure for the required displacement in time to deliver all of the drug in the reservoir chamber 420.

The processor(s) of the hypodermic needle device 400 (e.g., associated with the electronic units 432a and 432b) may be configured to control the pump 408 in order to transfer the specific dosage amount of the drug from the drug chamber 430 to the reservoir chamber 420. The pump 408 may be any suitable pump that transfers a precise volume of the drug with each stroke, with the total number of strokes performed by the pump 408 controllable by the processor(s). Thus, the processor(s) may operate the pump 408 until the specific dosage amount of the drug has been transferred to the reservoir chamber 420. Afterwards, the user may inject themselves with the needle assembly 404 and then dispense the drug by depressing the dose dispensing button 450, causing the specific dosage amount of the drug in the reservoir chamber 420 to be expelled out the needle assembly 404 secured to the reservoir container 425 (which, in some embodiments, may be part of the body 410) of the hypodermic needle device 400. In some embodiments, the processor(s) may operate the pump 408 to transfer the specific dosage amount of the drug within a specific tolerance, with some examples including transferring an amount within a 0.01%, 0.1%, 0.5%, 1%, 5%, or 10% tolerance of the specific dosage amount. For instance, if the pump 408 transferred 105 pL when the specific dosage amount is 100 pL, that would be within a 5% tolerance.

In some cases, the hypodermic needle device 400 may include a dosage dial 240 that may be adjusted prior to use (e.g., by turning the dial clockwise or counterclockwise) to alter the specific dosage amount dispensed by the hypodermic needle device 400. The setting of the dosage dial 440 may be used by the processor(s) in determining how much of the drug to transfer from the drug chamber 430 to the reservoir chamber 420. Thus, the setting of the dosage dial 440 may play an indirect role in the total number of strokes performed by the pump 408.

In some embodiments, there may be a fingerprint sensor disposed on the dose dispensing button 450 that serves as a mechanism for ensuring that the drug is being dispensed to the right person. For example, the processor(s) may monitor the fingerprint sensor for a fingerprint that matches the fingerprint profile of an authorized user. The fingerprint may need to be validated at multiple points in the dispensing process, such as prior to measuring out the specific dosage amount of the drug or prior to operation of the dose dispensing mechanism. For instance, if the correct fingerprint is not presented to sensor, then the pump 408 or any other valves present in the hypodermic needle device 400 may be shut off. As another example, if the correct fingerprint is not presented to sensor prior to the dispensing process, the mechanical or electronic actuation of the dispensing piston 402 may be prevented and no drug will be expelled from the reservoir chamber 420. In some embodiments, the fingerprint itself may be used to electronically drive the actuator associated with the dispensing piston 402 instead of the user depressing the dispensing button 450. The user may have to keep their finger on the fingerprint sensor throughout the dispensing process, as the dispensing piston 402 is actuated to expel all of the drug contained in the reservoir chamber 420.

To summarize the embodiment depicted in FIG. 4, a pump 408 is positioned between the drug chamber 430 and the reservoir chamber 420 and used to pump a specific dosage amount of the drug from the drug chamber 430 into the reservoir chamber 420. Once the drug is in the reservoir chamber 420, the user may inject themselves with the needle assembly 404 of the hypodermic needle device 400. The dose dispensing mechanism (e.g., the dispensing piston 402 mechanically coupled to the shaft 406) may be actuated in order to dispense all of the drug in the reservoir chamber 420 into the user.

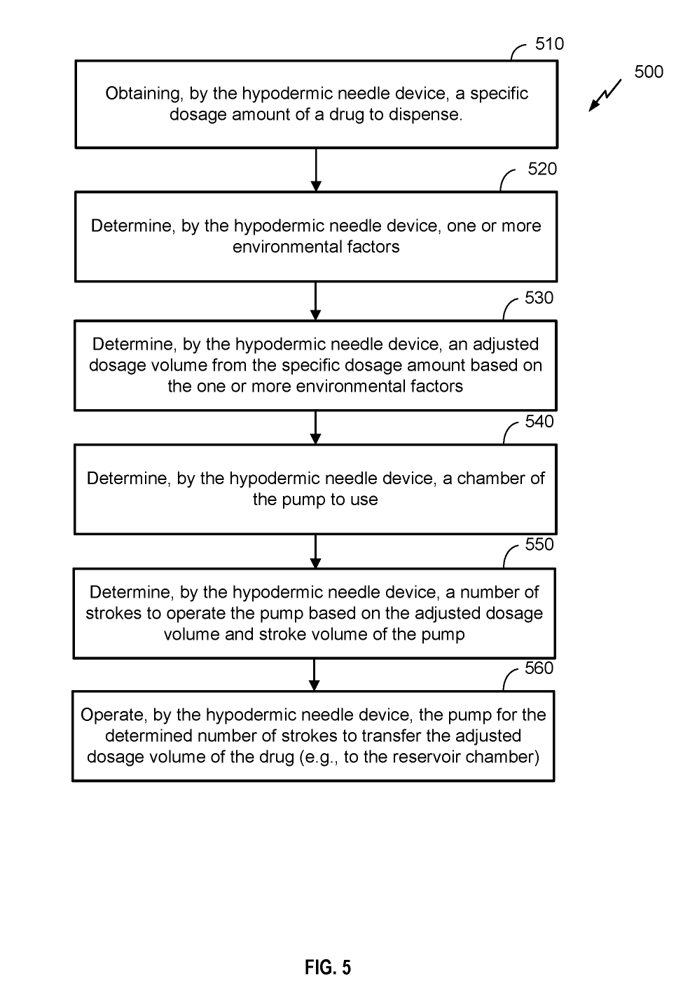

FIG. 5 is a flow diagram illustrating a method 500 of dispensing a drug with the embodiment of a hypodermic needle device illustrated in FIG. 4 (e.g., the hypodermic needle device 400).

It may be noted that, as with figures appended hereto, FIG. 5 is provided as a non-limiting example. Other embodiments may vary, depending on desired functionality. For example, the functional blocks illustrated in method 500 may be combined, separated, or rearranged to accommodate different embodiments. The method 500 may be performed by a hypodermic needle device. Means for performing the functionality of method 500 may include one or more components of the hypodermic needle device, including hardware and/or software components, as illustrated in FIG. 4 above. Hardware components may include analog and/or digital circuitry, including one or more processing units (microprocessors, digital signal processors (DSPs), etc.). A person of ordinary skill in the art will appreciate the various means by which the functions in method 500 may be performed.

At block 510, the hypodermic needle device may determine a specific dosage amount of a drug to dispense to a user. In some cases, the specific dosage amount may be pre-programmed into the hypodermic needle device, or it may be electronically communicated to the hypodermic needle device (e.g., via the communication link 120 or the communication network 150 in FIG. 1). In some cases, the sensor(s) and/or processor(s) of the hypodermic needle device may be configured to identify the drug being dispensed and determine a specific dosage amount based on the drug identified. In some cases, a user of the hypodermic needle device may set the specific dosage amount prior to use by adjusting the dosage dial of the hypodermic needle device (e.g., the dosage dial 440). The specific dosage amount may convey an amount of substance or chemical amount of the drug (e.g., a specific number of molecules of a drug), or the specific dosage amount may convey a volume of the drug to be dispensed under baseline conditions (e.g., at a baseline temperature and/or pressure).

In some embodiments, the hypodermic needle device may instead obtain a specific dosage amount (e.g., from a separate device). For example, the hypodermic needle device may offload determination of the specific dosage amount to a third-party device, such as a remote computer, mobile device, or smartphone that the hypodermic needle device is in electronic communication with. Thus, a user (e.g., such as a doctor or a patient attempting to self-administer the drug) may be able to set the specific dosage amount on a remote computer, mobile device, or smartphone before the hypodermic needle device is used to dispense that specific dosage amount.

Means for performing the functionality of block 510 may comprise, for example, a processor, such as processor 307 shown in FIG. 3, which may obtain the specific dosage amount from the setting of the dose control mechanism 304 or may obtain it from communicating with other devices via the communication interface 310 illustrated in FIG. 3, as well as the communication link 120 and/or communication network 150 shown in FIG. 1 and described above.

At block 520, the hypodermic needle device may determine one or more environmental factors. For example, the hypodermic needle device may measure a current ambient temperature or a current ambient pressure.

Means for performing the functionality of block 520 may comprise, for example, a processor, such as processor 307 shown in FIG. 3, communicating with the sensor(s) and actuator(s) 308. The sensor(s) may include temperature or pressure sensors that can report values to the processor 307 upon request.

At block 530, the hypodermic needle device may determine an adjusted dosage volume from the specific dosage amount based on the one or more environmental factors. For example, the density of the drug at various temperatures and/or pressures may be known. The density of the drug at the current temperature and pressure measured by the hypodermic needle device may be used to calculate an adjusted dosage volume of the drug that compensates for the current temperature and pressure, such that the specific dosage amount of the drug is being dispensed to the user regardless of the current temperature and pressure.

Means for performing the functionality of block 530 may comprise, for example, a processor, such as processor 307 shown in FIG. 3. The processor 307 may also perform this function by communicating with other devices via the communication interface 310 illustrated in FIG. 3, as well as the communication link 120 and/or communication network 150 shown in FIG. 1 and described above.

At block 540, the hypodermic needle device may determine a chamber of the pump to use (optional),In some cases, the pump may only have a single chamber and only one setting for how much volume is displaced in a single stroke of the pump. However, in other embodiments, the pump may have multiple chambers which allows for multiple settings for how much volume is displaced in a single stroke of the pump. For example, larger chambers may result in larger volumes being displaced in a single stroke of the pump. Thus, larger chambers may be used to dispense larger doses more quickly, while smaller chambers may be used for smaller doses. A multi-chambered pump may be used to manage different dosing requirements.

In some embodiments, the hypodermic needle device may consider the adjusted dosage volume and select a pump chamber to use that minimizes the number of strokes needed (in order to save time) while still allowing for the adjusted dosage volume to be accurately displaced. In other words, the hypodermic needle device may not necessarily select the largest chamber available. As an example, if the adjusted dosage volume is 250 pL and there exists a chamber that results in 100 pL being displaced in a single stroke of the pump, the hypodermic needle device may avoid using that chamber—even though using that chamber would displace the total volume in three strokes, it would be difficult to measure out exactly 250 pL (since two strokes would be under, while three strokes would be over).

Means for performing the functionality of block 540 may comprise, for example, a processor, such as processor 307 shown in FIG. 3.

In some embodiments, the hypodermic needle device may consider the adjusted dosage volume and select a pump chamber to use that minimizes the number of strokes needed (in order to save time) while still allowing for the adjusted dosage volume to be accurately displaced. In other words, the hypodermic needle device may not necessarily select the largest chamber available. As an example, if the adjusted dosage volume is 250 pL and there exists a chamber that results in 100 pL being displaced in a single stroke of the pump, the hypodermic needle device may avoid using that chamber—even though using that chamber would displace the total volume in three strokes, it would be difficult to measure out exactly 250 pL (since two strokes would be under, while three strokes would be over).

At block 550, the hypodermic needle device may determine the number of strokes needed to operate the pump based on the adjusted dosage volume and stroke volume of the pump (e.g., the stroke volume of the chamber selected). This process may involve dividing the adjusted dosage volume by the stroke volume.

Means for performing the functionality of block 550 may comprise, for example, a processor, such as processor 307 shown in FIG. 3.

At block 560, the hypodermic needle device may operate the pump for the determined number of strokes (using the selected chamber of the pump, if multiple are available) in order to transfer the adjusted dosage volume of the drug. In some embodiments, the adjusted dosage volume of the drug is transferred from the drug chamber to the reservoir chamber. In such embodiments, the hypodermic needle device will stop the operation of the pump and the reservoir chamber will contain the adjusted dosage volume of the drug. The user can then inject themselves with the hypodermic needle device, which can then dispense the adjusted dosage volume of the drug that was transferred. For example, in some embodiments, the adjusted dosage volume of the drug is measured out and stored in the reservoir chamber of the hypodermic needle device. The user injects the needle assembly of the hypodermic needle device into themselves. Once injected, the device may dispense the adjusted dosage volume of the drug that is contained in the reservoir chamber. In some embodiments, this may involve the user depressing a dose dispensing button (e.g., the dose dispensing button 450) at the end of the hypodermic needle device to force the drug in the reservoir chamber to flow out of the hollow needle of the needle assembly and into themselves.

In some embodiments, the user may first inject the needle assembly of the hypodermic needle device into themselves prior to the transfer occuring. Once the needle assembly has been injected into the user, the hypodermic needle device may operate the pump for the determined number of strokes (using the selected chamber of the pump, if multiple are available) in order to transfer the adjusted dosage volume of the drug from the drug chamber directly into the user via the needle assembly. Once the injection is complete and the pump has dispensed the specific dosage amount of the drug, the hypodermic needle device may notify the user (e.g., through a display or an audio signal) that the injection is complete and the hypodermic needle device can may be removed from the user's body.

Means for performing the functionality of block 560 may comprise, for example, a processor, such as processor 307 shown in FIG. 3, controlling the pump 312 for a number of strokes. It may also include the reservoir chamber 420 shown in FIG. 4, the needle assembly 230 shown in FIG. 2 or the needle assembly 404 shown in FIG. 4, and/or the dose dispensing button 450 shown in FIG. 4, which can be connected to a plunger that forces the drug out of the reservoir chamber.

FIG. 6 is a simplified cross-sectional diagram of an embodiment of a hypodermic needle device 600. The hypodermic needle device 600 may have a body 610 for housing dose dispensing and dose control mechanisms, including electrical and mechanical components, to ensure that a proper dosage of the drug is dispensed. In the figure, these components are shown as electronic unit 632, which may include sensors, actuators, processors, as well as communication interface circuitries. The sensors may collect operation information including, for example, the temperature and pressure surrounding the hypodermic needle device 600, the temperature and pressure of the drug (e.g., in a drug container 635), and so forth. The processors may be able to collect operation information from the sensors and consider that information in order to execute certain decisions (e.g., to send instructions for controlling the actuators).

The hypodermic needle device 600 may have a drug container 635 containing a drug chamber 630. The drug chamber 630 may contain a drug to be delivered to a user of the hypodermic needle device 600 via a needle assembly 604. The dose dispensing mechanism of the hypodermic needle device 600 may include a pump 608. The pump 608 may be configured to expel a drug contained in the drug chamber 630 out through the needle assembly 604.

The processor(s) of the hypodermic needle device 600 (e.g., associated with the electronic unit 630) may be configured to control the pump 608 in order to transfer the specific dosage amount of the drug from the drug chamber 630 to the user (e.g., once the user has injected themselves with the needle assembly 604). The pump 608 may be any suitable pump that transfers a precise volume of the drug with each stroke, with the total number of strokes performed by the pump 608 controllable by the processor(s). Thus, the user may first inject themselves with the needle assembly 604. The user may then operate the dose dispensing button 650, which may cause the processor(s) to initiate operation of the pump 608 until the specific dosage amount of the drug has been transferred from the drug chamber 630 to the user via the needle assembly 604.

In some cases, the hypodermic needle device 600 may include a dosage dial 640 that may be adjusted prior to use (e.g., by turning the dial clockwise or counterclockwise) to alter the specific dosage amount dispensed by the hypodermic needle device 600. The setting of the dosage dial 640 may be used by the processor(s) in determining how much of the drug to transfer from the drug chamber 630 to the user in a single dose. Thus, the setting of the dosage dial 640 may play an indirect role in the total number of strokes performed by the pump 608.

In some embodiments, the hypodermic needle device 600 may have a display (e.g., a LED) or audio generator (e.g., a speaker or beep generator) to inform the user when the injection is complete and the pump 608 has finished dispensing the specific dosage amount of the drug into the user. This allows the needle assembly 604 to be in the user for the minimum amount of time necessary.

In some embodiments, there may be a fingerprint sensor disposed on the dose dispensing button 650 that serves as a mechanism for ensuring that the drug is being dispensed to the right person. For example, the processor(s) may monitor the fingerprint sensor for a fingerprint that matches the fingerprint profile of an authorized user. The fingerprint may need to be validated prior to operation of the pump 608. For instance, if the correct fingerprint is not presented to sensor, then the pump 608 or any other valves present in the hypodermic needle device 600 may be shut off and cannot be used to dispense the drug from the drug chamber 630.

To summarize the embodiment depicted in FIG. 6, a pump 608 is positioned between a drug chamber 630 (e.g., a vial containing medicinal fluid) and a needle assembly 604. The pump 608 is similar to an insulin/infusion pump and is used to expel a specific dosage amount of a drug out of the drug chamber 630 through the needle assembly 604. Thus, the user may inject themselves with the needle assembly 604 and the pump 608 may be operated in order to precisely dispense the specific dosage amount of the drug into the user.

Additional functions may be performed, depending on desired functionality. For instance, some embodiments may further include sending, to another device, an indication of the determination of the effectiveness of how the drug was dispensed with the hypodermic needle device.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

With reference to the appended figures, components that may comprise memory may comprise non-transitory machine-readable media. The term “machine-readable medium” and “computer-readable medium” as used herein, refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including but not limited to, non-volatile media, volatile media, and transmission media. Common forms of computer-readable media include, for example, magnetic and/or optical media, any other physical medium with patterns of holes, a RAM, a PROM, EPROM, a FLASH-EPROM, any other memory chip or cartridge, a carrier wave as described hereinafter, or any other medium from which a computer may read instructions and/or code.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein may be embodied in hardware and/or software. Also, technology evolves and, thus, many of the elements are examples that do not limit the scope of the disclosure to those specific examples.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

Therefore, it is intended that claimed subject matter not be limited to the particular examples disclosed, but that such claimed subject matter may also include all aspects falling within the scope of appended claims, and equivalents thereof.

What is claimed is:

1. A method of dispensing a drug with a hypodermic needle device, the method comprising:
obtaining, by the hypodermic needle device, a specific dosage amount of the drug to dispense;
determining, by the hypodermic needle device, one or more environmental factors, wherein one of the one or more environmental factors includes an ambient temperature;
determining, by the hypodermic needle device, an adjusted dosage volume from the specific dosage amount based on the one or more environmental factors;
determining, by the hypodermic needle device, a number of strokes to operate a pump of the hypodermic needle device based on the adjusted dosage volume and a stroke volume of the pump; and
operating, by the hypodermic needle device, the pump for the determined number of strokes to transfer the adjusted dosage volume of the drug.

2. The method of claim 1, wherein operating the pump for the determined number of strokes serves to transfer the adjusted dosage volume of the drug into a reservoir chamber of the hypodermic needle device.

3. The method of claim 2, further comprising:
dispensing, by the hypodermic needle device, the adjusted dosage volume of the drug in the reservoir chamber.

4. The method of claim 1, wherein operating the pump for the determined number of strokes serves to dispense the adjusted dosage volume of the drug from the hypodermic needle device.

5. The method of claim 1, wherein the pump is a multi-chambered pump having a plurality of chambers.

6. The method of claim 5, further comprising:
selecting, by the hypodermic needle device, a chamber of the plurality of chambers of the multi-chambered pump based on the adjusted dosage volume and a stroke volume associated with the chamber.

7. The method of claim 6, wherein the chamber of the plurality of chambers of the multi-chambered pump is selected to minimize the number of strokes.

8. The method of claim 1, wherein the one or more environmental factors includes a density of the drug at a current temperature.

9. The method of claim 1, wherein the one or more environmental factors includes a density of the drug at a current pressure.

10. The method of claim 1, wherein the specific dosage amount of the drug is obtained from a third-party device.

11. A hypodermic needle device comprising:
a drug cartridge containing a drug;
a reservoir configured to hold the drug prior to injection;
a dose dispensing mechanism configured to transfer an amount of the drug contained in the drug cartridge to the reservoir;
a sensor for measuring one or more environmental factors, wherein one of the one or more environmental factors includes an ambient temperature;
a processor;
a non-transitory computer readable memory storing instructions that, when executed by the processor, cause the processor to:
determine a specific dosage amount of the drug to dispense;
determine the one or more environmental factors from the sensor;
determine an adjusted dosage volume from the specific dosage amount based on the one or more environmental factors; and
operate the dose dispensing mechanism to transfer the adjusted dosage volume of the drug.

12. The device of claim 11, wherein operating the dose dispensing mechanism serves to transfer the adjusted dosage volume of the drug from the drug cartridge into the reservoir.

13. The device of claim 12, wherein the instructions, when executed by the processor, further cause the processor to:
dispense the adjusted dosage volume of the drug in the reservoir.

14. The device of claim 11, wherein operating the dose dispensing mechanism serves to dispense the adjusted dosage volume of the drug from the hypodermic needle device.

15. The device of claim 11, wherein the dose dispensing mechanism is an electronically-controlled pump.

16. The device of claim 15, wherein the instructions, when executed by the processor, further cause the processor to:
determine a number of strokes to operate the pump based on the adjusted dosage volume and a stroke volume of the pump; and
operate the pump for the determined number of strokes to transfer the adjusted dosage volume of the drug.

17. The device of claim 13, wherein the hypodermic needle device further comprises a needle configured for dispensing the drug contained in the reservoir.

18. The device of claim 11, wherein the one or more environmental factors includes a density of the drug at a current temperature.

19. The device of claim 11, wherein the one or more environmental factors includes a density of the drug at a current pressure.

20. The device of claim 11, wherein determining the specific dosage amount of the drug includes obtaining the specific dosage amount from a third-party device.

21. A hypodermic needle device comprising:
means for obtaining a specific dosage amount of the drug to dispense;
means for determining one or more environmental factors, wherein one of the one or more environmental factors includes an ambient temperature;
means for determining an adjusted dosage volume from the specific dosage amount based on the one or more environmental factors; and
means for transferring the adjusted dosage volume of the drug.

22. The device of claim 21, further comprising:
means for determining a number of strokes to operate a pump of the hypodermic needle device based on the adjusted dosage volume and a stroke volume of the pump; and
means for operating the pump for the determined number of strokes to transfer the adjusted dosage volume of the drug.

23. The device of claim 22, wherein the pump is a multi-chambered pump having a plurality of chambers.

24. The device of claim 23, further comprising:
means for selecting a chamber of the plurality of chambers of the multi-chambered pump based on the adjusted dosage volume and a stroke volume associated with the chamber.

25. The device of claim 21, wherein the one or more environmental factors includes a density of the drug at a current temperature.

26. The device of claim 21, wherein the one or more environmental factors includes a density of the drug at a current pressure.

27. The device of claim 21, wherein the specific dosage amount of the drug is obtained from a third-party device.

28. A non-transitory computer readable medium containing instructions that, when executed by a processor, cause the processor to:
obtain a specific dosage amount of the drug to dispense;
determine one or more environmental factors, wherein one of the one or more environmental factors includes an ambient temperature;
determine an adjusted dosage volume from the specific dosage amount based on the one or more environmental factors;
determine a number of strokes to operate a pump of the hypodermic needle device based on the adjusted dosage volume and a stroke volume of the pump; and
operate the pump for the determined number of strokes to transfer the adjusted dosage volume of the drug.

29. The non-transitory computer readable medium of claim 28, wherein the one or more environmental factors includes a density of the drug at a current temperature.

30. The non-transitory computer readable medium of claim 28, wherein the one or more environmental factors includes a density of the drug at a current pressure.
